(19) United States
(12) Patent Application Publication
(10) Pub. No.: US 2023/0113701 A1
(43) Pub. Date: Apr. 13, 2023

(54) MAGNETIC SIGNAL DETECTION CHIP, DETECTION CARD, NUCLEIC ACID DETECTION DEVICE, AND METHOD OF DETECTING COMPOSITION CONTAINING TARGET DNA FRAGMENT IN CHEMICAL FLUID

(71) Applicant: TDK CORPORATION, Tokyo (JP)

(72) Inventors: Jacky HO, Nancheng Dongguan (CN); Jun De DENG, Nancheng Dongguan (CN)

(73) Assignee: TDK CORPORATION, Tokyo (JP)

(21) Appl. No.: 17/914,396

(22) PCT Filed: Mar. 30, 2021
(86) PCT No.: PCT/IB2021/052601

§ 371 Date: Sep. 26, 2022

(30) Foreign Application Priority Data
Mar. 31, 2020 (CN) 202010245193.2

(51) Int. Cl. C12Q 1/6806; G01N 27/74; B01L 3/00

(52) U.S. Cl. CPC C12Q 1/6806; G01N 27/745; B01L 3/502761; B01L 2300/0816; B01L 2200/0647; B01L 2400/0487; B01L 2200/026; B01L 2300/0681; B01L 2300/0861

(57) ABSTRACT

Provided is a magnetic signal detection chip. Each flow sensing component thereof includes lead portions and notch portion(s) located between the adjacent lead portions, wherein the notch portion(s) is(are) located in the flow path and at least a part of each lead portion adjacent to the notch portion is exposed to the chemical fluid, so that the flow sensing component(s) is(are) conducted when the chemical fluid completely covers the notch portion(s). Thus one could regulate the processing steps according to the sensing results of the flow sensing component, which can avoid inaccurate detection results. Further provided is a detection card including the magnetic signal detection chip and further provided is a nucleic acid detection device including the detection card. Further provided is a method of detecting composition containing target DNA fragment in chemical fluid.

### TECHNICAL FIELD

The present disclosure relates to a technique for detecting the composition of a chemical substance by means of magnetic signals, in particular a magnetic signal detection chip, a detection card including the magnetic signal detection chip, and a nucleic acid detection device including the detection card, and also relates to a method of detecting composition containing target DNA fragment in chemical fluid by means of the nucleic acid detection device.

### BACKGROUND

In the prior art, a detection card including a magnetic signal detection chip is used to quantitatively detect the composition of chemical substances by means of the giant magneto resistance effect. For example, in a Chinese invention patent application with the publication number CN109917139A titled with “GMR chip and magnetic sensitive immunodetection card including the same” has disclosed a GMR chip and a magnetic sensitive immunodetection card for protein detection. However, in the prior art represented by the aforementioned Chinese invention patent application, a function of sensing the flow status of the chemical fluid flowing through the GMR chip is lacked, and it is also impossible to determine whether the chemical fluid flows throughout the whole sensing region of the GMR chip. In the prior art, the processing steps of the GMR chip are designed to be implemented at predetermined intervals, rather than being implemented based on real-time feedbacks of the flow status of the chemical fluid. In this case, the sensing results of the GMR chip in the prior art will be inaccurate, when the flow of the chemical fluid is delayed or other problems (e.g. non-smooth or leaking) occur.

### SUMMARY

The present disclosure was made in view of the above-mentioned defects of the prior art. An object of the present disclosure is to provide a novelty magnetic signal detection chip, which can sense and feedback the flow status of the chemical fluid flowing through the magnetic signal detection chip in real time. Another object of the present disclosure is to provide a detection card including the above-mentioned magnetic signal detection chip and a nucleic acid detection device including the detection card. Further object of the present disclosure is to provide a method of detecting composition containing target DNA fragment in chemical fluid by means of the above nucleic acid detection device, which can accurately detect the composition containing the target DNA fragment.

In order to obtain the above objects, the present disclosure includes the following technical solutions.

This disclosure provides a magnetic signal detection chip, comprising:

a plurality of chemical composition sensing units, which are disposed inside the flow path of the magnetic signal detection chip, through which chemical fluid flows; and

one or more flow sensing component(s), which extend(s) to the outside of the flow path across the flow path, wherein the flow sensing component comprises lead portions and notch portion(s) located between the adjacent lead portions, wherein the notch portion(s) is(are) located in the flow path and at least a part of each lead portion adjacent to the notch portion is exposed to the chemical fluid, so that the flow sensing component(s) is(are) conducted when the chemical fluid completely covers the notch portion(s).

Preferably, the lead portions and the notch portion(s) as a whole extends substantially in an orthogonal direction orthogonal to the flow direction of the chemical fluid.

More preferably, the lead portion comprises interruption/conducting ends at the notch portion, and

in the orthogonal direction, the two outermost interruption/conducting ends of the flow sensing component are located at the boundaries of the flow path or are respectively located at inner side of the corresponding boundary.

More preferably, the flow sensing component comprises only one notch portion, wherein the notch portion is located in the central part in the extending direction of the flow sensing component.

More preferably, the flow sensing component comprises a plurality of notch portions spaced apart from each other, and wherein the plurality of notch portions are substantially uniformly distributed in the flow sensing component in the extending direction of the flow sensing component.

More preferably, the plurality of chemical composition sensing units are arranged in multiple rows, and the arrangement direction of the chemical composition sensing units in each row is along the flow direction of the chemical fluid, and each of the notch portions in the flow sensing component are respectively located in a row in which the chemical composition sensing units are arranged.

More preferably, the flow sensing component further comprises output pads at both ends thereof in an orthogonal direction orthogonal to the flow direction and, wherein the output pads are connected to the lead portions and are disposed outside the flow path.

More preferably, multiple flow detection components are provided, and wherein the multiple flow detection components are arranged in the way of being spaced by the chemical composition sensing units in the flow direction of the chemical fluid,

wherein two of the multiple flow detection components are respectively disposed at two ends of the flow path in the flow direction, and/or one of the multiple flow detection components is disposed at the central part of the flow path in the flow direction.

More preferably, one flow detection component is provided, which is disposed at the downstream end of the flow path in the flow direction of the chemical fluid.

This disclosure further provides a detection card, comprising the magnetic signal detection chip according to any one of the above technical solutions.

This disclosure further provides a nucleic acid detection device, comprising the detection card of the above technical solution.

This disclosure further provides a method of detecting composition containing target DNA fragment in chemical fluid, comprising the following steps:

chemical fluid flowing step, in which during the chemical fluid flowing through a nucleic acid detection device, the chemical fluid flows backwards and forwards in the flow path of a magnetic signal detection chip of the nucleic acid detection device, thereby the chemical fluid completely covers the flow path, so that the composition is fixed to the chemical component sensing unit of the magnetic signal detection chip; and

detecting step, in which the composition containing the target DNA fragment is detected by the magnetic signal detection chip.

Preferably, the nucleic acid detection device is the nucleic acid detection device as describe above.

More preferably, the nucleic acid detection device is provided with an inlet portion in communication with the flow path of the magnetic signal detection chip, wherein the chemical fluid flows into the flow path via the inlet portion, and wherein the cross-sectional area of the inlet portion gradually increases towards the flow path.

More preferably, the width of the part of the inlet portion connected to the flow path is the same as the width of the flow path.

More preferably, the nucleic acid detection device is provided with an outlet portion in communication with the flow path of the magnetic signal detection chip, wherein the chemical fluid flows away from the flow path via the outlet portion, and wherein the cross-sectional area of the outlet portion gradually decreases from the flow path.

More preferably, the width of the part of the outlet portion connected to the flow path is the same as the width of the flow path.

More preferably, the inlet portion and the outlet portion are configured to be symmetrical relative to the centerline of the flow path, wherein the centerline extends along a direction perpendicular to the flow direction of the chemical fluid.

Upon the above technical solutions, the present disclosure provides a novelty magnetic signal detection chip, a detection card including the magnetic signal detection chip, and a nucleic acid detection device including the detection card. In the magnetic signal detection chip, the flow sensing component extends across the flow path. The flow sensing component includes lead portions and notch portion(s) located between adjacent lead portions. The notch portion(s) is(are) located in the flow path and at least a part of each lead portion adjacent to the notch portion is exposed to the chemical fluid, so that the flow sensing 1o component is conducted, when the chemical fluid completely covers the notch portion(s). In this way, when the chemical fluid flows through the flow sensing component, the flow sensing component can sense the flow status of the chemical fluid and can feed back sensing results to the magnetic signal detection chip or the detection card in real time, thereby the magnetic signal detection chip or detection card according to the present disclosure can regulate the processing steps according to the sensing results of the flow sensing component, which can avoid inaccurate detection results caused by the processing steps in the prior art performed at predetermined intervals regardless of the flow status of the chemical fluid.

In addition, the present disclosure further provides a method of detecting composition containing target DNA fragment in chemical fluid by means of the nucleic acid detection device described above. In this method, the chemical fluid flows backwards and forwards inside the flow path of the magnetic signal detection chip, so that the chemical fluid can completely cover the entire flow path. In this way, the situation that the composition is not fixed to the corresponding chemical component sensing unit can be fully avoided, so that the composition containing the target DNA fragment can be accurately detected.

### BRIEF INTRODUCTION OF THE DRAWINGS

FIG. 1A is a schematic view showing the structure of a magnetic signal detection chip according to an embodiment of the present disclosure, in which the hollow double-headed arrows indicate the flow direction of the chemical fluid flowing through the sensing region of the magnetic signal detection chip; FIG. 1B is a schematic view showing the structure of the flow sensing component of the magnetic signal detection chip shown in FIG. 1A.

FIG. 2 is a schematic view showing the structure of a modified example of the flow sensing component in FIG. 1B.

FIG. 3 is a schematic view showing a nucleic acid detection device according to the present disclosure including the magnetic signal detection chip shown in FIG. 1A.

FIG. 4 is an enlarged schematic view showing a partial structure of the nucleic acid detection device in FIG. 3, in which the nucleic acid detection unit and its surrounding structure are shown.

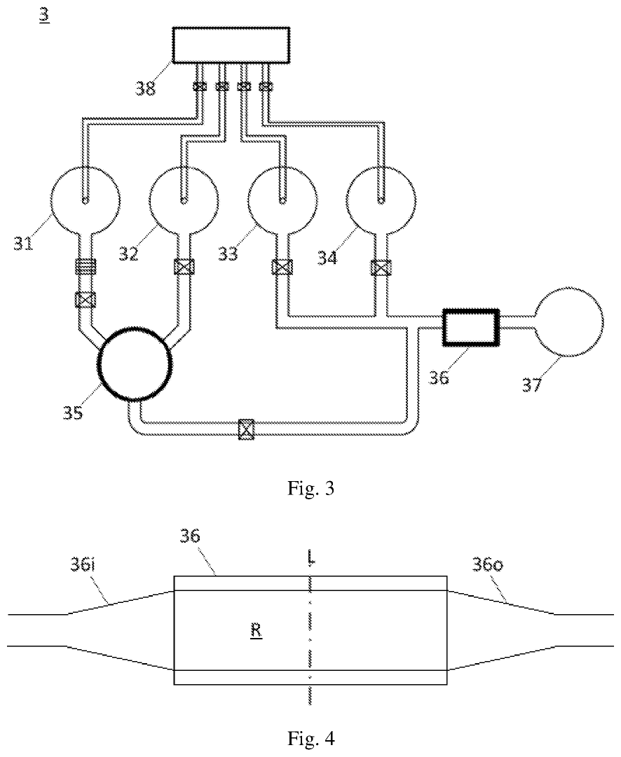

### LIST OF REFERENCE SIGNS

11 chemical composition sensing unit
12 first output pad
13 ground pad
14 lead
2 flow sensing component
21 lead portion
21a interruption/conducting end
22 notch portion
23 second output pad
3 nucleic acid detection device
31 sample chamber
32 PCR solution chamber
33 magnetic beads chamber
34 hybridization solution chamber
35 PCR reaction chamber
36 nucleic acid detection unit
36i inlet portion
36o outlet portion
L centerline
37 waste tank
38 pump
F flow direction
X centerline
R flow path

### DETAILED EMBODIMENTS

Exemplary embodiments of the present disclosure will be described below with reference to the drawings. It should be understood that these specific descriptions are only intended to teach those skilled in the art how to practice the present disclosure, and are not intended to be exhaustive of all possible ways of carrying out the present disclosure or to limit the scope of the present disclosure.

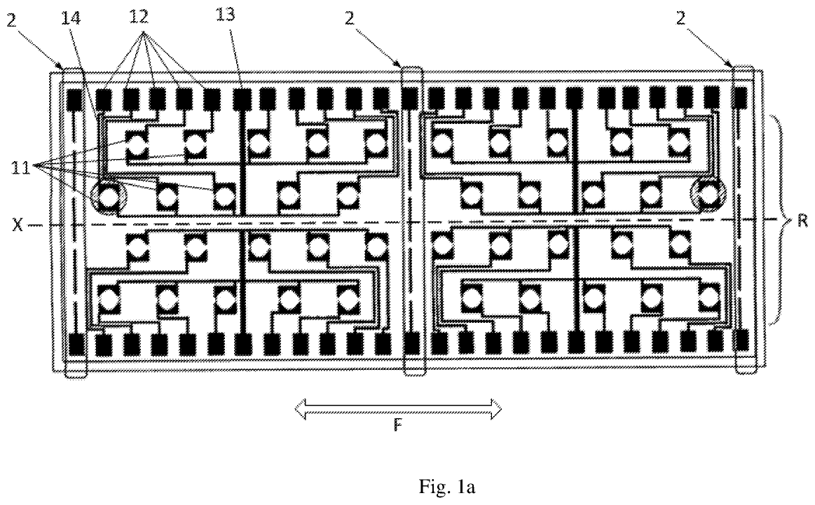

It should be noted that in the present disclosure, “both sides” refer to both sides of the first centerline X of the magnetic signal detection chip, while “one side” refers to the upper side in FIGS. 1A and 1B and “the other side” refers to the lower side in FIGS. 1A and 1B; “outer side” refers to the side far away from the centerline X and “inner side” refers to the side close to the centerline X.

The structure of the magnetic signal detection chip according to an embodiment of the present disclosure will be described below with reference to the drawings in the specification.

(Structure of the Magnetic Signal Detection Chip According to an Embodiment of the Present Disclosure)

As shown in FIG. 1A, the magnetic signal detection chip according to an embodiment of the present disclosure includes a plurality of chemical composition sensing units 11, a plurality of first output pads 12, a plurality of ground pads 13, a plurality of leads 14, and three flow sensing components 2 provided on the matrix of the magnetic signal detection chip.

Specifically, in the present embodiment, a plurality of chemical composition sensing units 11 are disposed inside the flow path R of the magnetic signal detection chip, and are used to sense the magnetic beads in the chemical fluid flowing through the flow path R, so as to quantitatively analyze the precise content of the components to be detected in the chemical fluid. The flow path R may be either a flow path in which the chemical fluid flows from the left side in FIG. 1A toward the right side in FIG. 1A or a flow path in which the chemical fluid flows from the right side in FIG. 1A toward the left side in FIG. 1A, wherein the flow path R may be defined by the counter structure of the detection card including the magnetic signal detection chip. In FIG. 1A, the range of the flow path R on both sides is indicated by angle brackets.

In this embodiment, the chemical composition sensing units 11 are arranged in four rows, wherein the extending direction of each row is along the flow direction F of the chemical fluid, and the sensing units 11 in one row are staggered from the sensing units 11 in an adjacent row in the flow direction F, thereby facilitating the routing of the leads 14 drawn from the chemical composition sensing units 11. Further, the chemical composition sensing units 11 are divided into four groups, and the number of the chemical composition sensing units 11 in each group is equal.

In this embodiment, the number of the first output pads 12 is equal to the number of the chemical composition sensing units 11, wherein the first output pads 12 are connected with the sensing units 11 in one-to-one mapping via the leads 14, so that the signals generated by the chemical composition sensing units 11 is outputted outside of the magnetic signal detection chip. The plurality of first output pads 12 are disposed outside the flow path R, so that the plurality of first output pads 12 are not in contact with the chemical fluid. In the present disclosure, different from the prior art, the plurality of first output pads 12 are located on both sides of the flow path R. Taking the centerline X of the flow path R extending along the flow direction F of the chemical fluid as a reference, the chemical composition sensing units 11 on one side and the first output pads 12 on the one side are respectively connected via the leads 14, while the chemical composition sensing units 11 on the other side and the first output pads 12 on the other side are respectively connected via the leads 14. In this way, not only the size of the magnetic signal detection chip can be reduced, but also the length of the leads 14 can be shortened when each chemical composition sensing unit 11 is connected to the first output pad 12 that is close to it via the lead 14, thereby the resistance of the leads 14 is reduced compared with the prior art.

Further, on both sides of the flow path R, the first output pads 12 are respectively arranged in one row, wherein the direction of the row extends along the flow direction F. In this way, compared with the solution that the first output pads 12 being arranged in multiple rows on one side in the prior art, the size of the magnetic signal detection chip can be further reduced and the length of the leads 14 can be further shortened. Further, the number of the first output pads 12 on one side of the flow path R is equal to the number of the first output pads 12 on the other side of the flow path R. In this way, the problems in routing caused by too dense arrangement of the first output pads 12 on one side can be avoided. Further, in the flow direction F, the region where the first output pads 12 are disposed does not exceed the region where the chemical composition sensing units 11 are disposed. In this way, it can be ensured that the size of the magnetic signal detection chip in the flow direction F will not become larger due to the excessive extension of the disposal region of the first output pads 12.

Further, corresponding to the chemical composition sensing unit 11 being divided into four groups, in this embodiment, the first output pads 12 are also divided into four groups. Each group of first output pads 12 is respectively connected to a group of chemical composition sensing units 11, and each group of first output pads 12 is disposed at a position close to the corresponding group of chemical composition sensing units 11, so that the length of the leads 14 between the first output pads 12 and the chemical composition sensing units 11 can be further shortened.

In this embodiment, each ground pad 13 is provided corresponding to a plurality of chemical composition sensing units 11 and is shared by the plurality of chemical composition sensing units 11, and the ground pad 13 is used for grounding rather than outputting signals of the chemical composition sensing unit 11 to outside. Similar to the first output pads 12 described above, the ground pads 13 are disposed outside the flow path R and each ground pad 13 is connected to a plurality of chemical composition sensing units 11 via leads 14. Corresponding to the chemical composition sensing unit 11 being divided into four groups, in this embodiment, four ground pads 13 are provided. One ground pad 13 is only connected to a group of chemical composition sensing units 11 via leads 14. In addition, the ground pad 13 corresponding to a group of chemical composition sensing units 11 and the first output pads 12 corresponding to the group of chemical composition sensing units 11 are arranged in one row, so that the size of the magnetic signal detection chip would not become larger due to the disposal of the ground pads 13.

In this embodiment, one end of each lead 14 is connected to a chemical composition sensing unit 11, and the other end of each lead 14 is connected to a first output pad 12 or a ground pad 13. Corresponding to the chemical composition sensing units 11 being divided into four groups, in this embodiment, the routing of the leads 14 is also divided into four parts, and the leads 14 should be routed as short as possible without affecting the normal operation of other components.

The structure of the flow sensing component 2 of the magnetic signal detection chip in FIG. 1A will be described below.

(Structure of Flow Sensing Component 2 of Magnetic Signal Detection Chip According to an Embodiment of the Present Disclosure)

In this embodiment, as shown in FIG. 1A, three flow sensing components 2 are arranged in the flow direction F of the chemical fluid with the groups of chemical component sensing units 11 therebetween, i.e. two flow sensing components 2 are disposed at both ends of the flow path R in the flow direction F and one flow sensing component 2 is disposed in the central part of the flow path R in the flow direction F. Each flow sensing component 2 extends to both sides of the flow path R across the flow path R. Specifically, as shown in FIG. 1B, each flow sensing component 2 includes a plurality of lead portions 21, a plurality of notch portions 22 located between adjacent lead portions 21, and second output pads 23 connected to the lead portions 21 located on both sides. The lead portions 21 and the notch portions 22 as a whole extend substantially in an orthogonal direction orthogonal to the flow direction F.

Further, the lead portions 21 are made of metal. The plurality of lead portions 21 located in the middle along the extending direction of the flow sensing component 2 are all provided inside the flow path R, and these lead portions 21 may be entirely exposed to the chemical fluid or the parts of the lead portions 21 adjacent to the notch portions 22 may be exposed to chemical fluid. In this way, when the chemical fluid completely covers the notch portions 22, the lead portions 21 adjacent to the notch portions 22 are conducted to each other. In addition, the lead portions 21 include interruption/conducting ends at the notch portions 22. In the orthogonal direction orthogonal to the flow direction F, the two outermost interruption/conducting ends 21a of each flow sensing component 2 are located at the boundaries on both sides or located inside of the corresponding boundaries. In this way, it can be ensured that the entire flow sensing component 2 can be conducted under the condition that the chemical fluid completely covers the notch portions 22.

Further, a plurality of notch portions 22 are disposed inside the flow path R with the lead portions 21 therebetween, wherein no lead is provided at the notch portions 22, so that the lead portions 21 adjacent to the notch portions 22 are disconnected at the notch portions 22. In this embodiment, the plurality of notch portions 22 are substantially uniformly distributed in the flow sensing component 2 in the extending direction of the flow sensing component 2. More specifically, in this embodiment, four notch portions 22 are provide in each flow sensing component 2, and each notch portion 22 of the flow sensing components 2 is located in the row in which the chemical component sensing units 11 are arranged. In this way, it can be reliably ensured that the chemical fluid have surely flowed across each row of the chemical component sensing units 11 when the flow sensing components 2 are conductive.

Further, the second output pads 23 are located at both ends of the flow sensing component 2 in the orthogonal direction orthogonal to the flow direction F, and the second output pads 23 are connected to the lead portions 21. The second output pads 23 are provided outside the flow path R, and therefore are not in contact with the chemical fluid. In this way, when the flow sensing component 2 is conductive, a corresponding signal can be outputted to the magnetic signal detection chip via the second output pads 23, for example.

Upon adopting the flow sensing components 2 having the above-mentioned structure, when the chemical fluid completely covers each notch portion 22 of the flow sensing components 2, the flow sensing components 2 are conducted and output corresponding signals, thereby real-time sensing and feeding back the flow status of the chemical fluid at the location of the flow sensing component 2 is possible, so that the magnetic signal detection chip can regulate the precise implementation timing of each processing step according to the information fed back by the flow sensing component 2. In addition, when the flow sensing components 2 having the above structure are conducted, it can also be ensured that the chemical fluid have flowed across each chemical component sensing unit 11, so that an inaccurate sensing result can be avoided, which is caused by that the chemical fluid does not flow across a certain chemical component sensing unit 11.

The structure of the magnetic signal detection chip according to an embodiment of the present disclosure and the structure of the flow sensing component 2 of such magnetic signal detection chip are described above. The structure of a modified example of the flow sensing component 2 of FIG. 1B will be described below.

(Structure of a Modified Example of the Flow Sensing Component)

The basic structure of the flow sensing component 2 according to the modified example of the present disclosure is similar to the basic structure of the flow sensing component 2 shown in FIG. 1B, and the differences between the two will be described below.

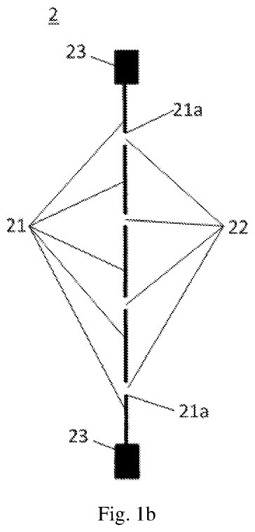

As shown in FIG. 2, in this modified example, the flow sensing component 2 includes only one notch portion 22, which is located at the center in the extending direction of the flow sensing component 2. Obviously, the notch portion 22 in this modified example is longer than each notch portion 22 of the flow sensing component 2 in FIG. 1B. When the conductivity of the chemical fluid is sufficient to ensure the lead portions 21 adjacent to the notch portion 22 conductively connected with each other under the condition of such long notch portion 22, the structure of this modified example can be available. The flow sensing component 2 of this modified example can realize all the functions of the flow sensing component 2 in FIG. 1B and further can save processing costs.

In addition, the present disclosure also provides a detection card including the above-mentioned magnetic signal detection chip. The detection card according to the present disclosure includes not only the above-mentioned magnetic signal detection chip, but also a printed circuit board (not shown in the figure), wherein the magnetic signal detection chip is disposed on the printed circuit board.

The structure of a nucleic acid detection device according to an embodiment of the present disclosure will be described below.

(Structure of a Nucleic Acid Detection Device According to an Embodiment of the Present Disclosure)

The nucleic acid detection device 3 according to an embodiment of the present disclosure includes a DNA extraction and PCR reaction system, a magnetic signal detection system, a heating system, a filtration system, a pump system, and a valve system.

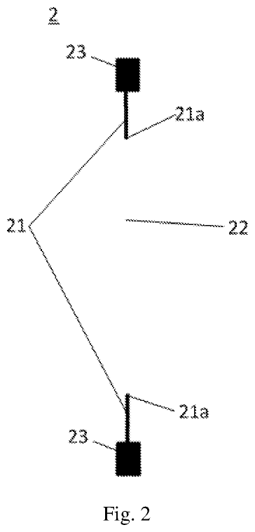

Specifically, as shown in FIG. 3, the DNA extraction and PCR reaction system includes a sample chamber 31, a PCR solution chamber 32 and a PCR reaction chamber 35, wherein the primers in the PCR solution chamber 32 are biotin-labeled primers.

The magnetic signal detection system includes a magnetic beads chamber 33, a hybridization solution chamber 34 and a nucleic acid detection unit 36. The nucleic acid detection unit 36 has a detection card including the above-mentioned magnetic signal detection chip, so that it can sense DNA-modified magnetic beads, wherein magnetic signals of the DNA-modified magnetic beads are converted into electrical signals.

The temperature change of the PCR reaction chamber 35 and the magnetic signal detection chip in the nucleic acid detection unit 36 can be controlled by the heating system (not shown in the figure).

Sample filtration can be achieved by means of the filtration system. The filtration system may include filter membranes with different pore sizes, such as 0.12 μm, 0.008 μm or 0.001 μm. Under the action of the pump system, the chemical fluid containing DNA flows through the filter membrane to remove cell debris and proteins therein.

Samples and PCR solution can be mixed under actions of the pump system and the valve system, as well as the PCR reaction products, hybridization solutions and magnetic beads can be mixed under actions of the pump system and the valve system. The pump system includes a pump 38, and the outlets of the pump system are respectively connected to the inlets of the sample chamber 31, the PCR solution chamber 32, the magnetic beads chamber 33, and the hybridization solution containing chamber 34 via four valves. The outlets of the sample chamber 31 and the PCR solution chamber 32 are respectively connected 1o to different inlets of the PCR reaction chamber 35. The outlets of the PCR reaction chamber 35, the magnetic beads chamber 33 and the hybridization solution chamber 34 are respectively connected to different inlets of the nucleic acid detection unit 36. A valve and a filter membrane are provided at the outlet of the sample chamber 31, a valve is provided at the outlet of the PCR solution chamber 32, and valves are provided at the outlets of the magnetic beads chamber 33 and the hybridization solution chamber 34.

Further, the nucleic acid detection unit 36 is connected to the waste tank 37 so as to introduce the waste into the waste tank 37.

When the nucleic acid detection device according to the present disclosure is used to detect composition containing target DNA fragment in the chemical fluid by means of the detection card, the pump 38 can be used to realize the backward and forward flow of the chemical fluid in the nucleic acid detection unit 36. Moreover, it is possible to prevent the chemical fluid from flowing back into the chambers by locating each valve of the valve system on position in the device and through the structures thereof.

A method of detecting composition containing target DNA fragment in the chemical fluid by means of the above-mentioned nucleic acid detection device will be described below.

(Method of Detecting the Composition Containing Target DNA Fragment in the Chemical Fluid According to the Present Disclosure)

In the method of detecting the composition containing target DNA fragment in the chemical fluid according to the present disclosure, the detection is performed by the nucleic acid detection device as described above, and the method includes a chemical fluid flowing step and a detecting step.

In the chemical fluid flowing step, when the chemical fluid flows through the nucleic acid detection device, a pump system (pump 38) can be used to make the chemical fluid flow backwards and forwards in the flow path R of the magnetic signal detection chip. In the process of backward and forward flow, the circles of backward and forward flows and the amplitude of each backward and forward flow can be adjusted according to requirements. Upon this kind of backward and forward flow, it can be ensured that the chemical fluid completely covers the entire flow path R of the magnetic signal detection chip, so that the compositions to be detected are fixed to the corresponding chemical composition sensing unit 11 (for example, fixed to the probes of the chemical composition sensing unit 11).

In the detecting step, the composition containing target DNA fragment is detected by the magnetic signal detection chip. Since the above-mentioned backward and forward flowing can fully avoid the situation that the composition to be detected is not fixed to the corresponding chemical component sensing unit 11, the accuracy of the detection result is reliable guaranteed.

It can provide excellent effect when the above-mentioned detection method is applied in the nucleic acid detection device 3 shown in FIG. 3 as described above, wherein the above-mentioned detection method is however not limited to use in this specific nucleic acid detection device, but can be applied to others as needed, especially regarding the chemical fluid flowing step in which the chemical fluid flows through the flow path in backward and forward flows.

In addition, in order to be cooperated with the backward and forward flow of the chemical fluid in the flow path of the magnetic signal detection chip, the nucleic acid detection device to which the above detection method is applied may preferably adopt following configurations. Taking the nucleic acid detection device shown in FIG. 3 as an example, the nucleic acid detection device 3 is provided with an inlet portion 36i in communication with the flow path R of the magnetic signal detection chip, wherein the chemical fluid flows into the flow path R via the inlet portion 36i. The cross-sectional area of the inlet portion 36i gradually increases towards the flow path R, and the width of the part of the inlet portion 36i connected to the flow path R is the same as the width of the flow path R. The nucleic acid detection device 3 is also provided with an outlet portion 36o in communication with the flow path R of the magnetic signal detection chip. The chemical fluid flows away from the flow path R via the outlet portion 36o. The cross-sectional area of the outlet portion 36o gradually decreases from the flow path R, and the width of the part of the outlet portion 36o connected to the flow path R is the same as the width of the flow path R.

It is further preferable that the inlet portion 36i and the outlet portion 36o are configured to be symmetrical relative to the centerline L of the flow path R, wherein the centerline L extends in a direction perpendicular to the flow direction of the chemical fluid.

It can provide an effective and reliable effect when the above-mentioned preferred structure is in combination with the backward and forward flow of the chemical fluid in the flow path. That is, it can assistant the chemical fluid to completely cover the flow path, and therefore it is beneficial to the fix of the composition containing the target DNA fragment to the corresponding sensing unit.

In summary, the present disclosure provides a novelty magnetic signal detection chip, a detection card including the magnetic signal detection chip, and a nucleic acid detection device including the detection card, which are not limited to those described in the above specific embodiments. The supplementary explanations will be provided as follows.

i. Although not described clearly in the above detailed embodiments, it should be understood that the magnetic signal detection chip according to the present disclosure is a magnetic induction element, wherein not only a giant magneto resistance chip (GMR chip) and tunnel magneto resistance chip (TMR chip) can be used, but also Hall chip can also be used. Preferably, the magnetic signal detection chip according to the present disclosure is a giant magneto resistance chip.

ii. Although not described clearly in the above detailed embodiments, it should be understood that the number of the chemical composition sensing units 11 of the magnetic signal detection chip can be determined upon the number of types of the components to be detected in the chemical fluid.

iii. Although the chemical composition sensing units 11 of the magnetic signal detection chip are divided into multiple groups in the above detailed embodiments, the present disclosure is not limited to this. As long as the chemical composition sensing units 11 are connected with the first output pads 12 on the same side, it is not necessary to divide the chemical composition sensing units 11 into multiple groups as described in the above detailed embodiments.

iv. Although not described in the above embodiment shown in FIG. 1A, it should be understood that in this embodiment, for each group of chemical component sensing units 11, the leads 14 of the chemical component sensing units 11 on the left side are routed on the left side of the group of chemical composition sensing units 11, while the leads 14 of the chemical composition sensing units 11 on the right side are routed on the right side of the group of chemical composition sensing units 11. Such routing of the leads 14 can prevent all the leads 14 from being routed together on the left side or on the right side, which results in an increase in the size of the magnetic signal detection chip.

v. Although not described in the above detailed embodiments, it can be provided in the technical solutions of the present disclosure that, an alarm indicating a delayed, non-smooth or leaking flow of the chemical fluid will be issued, if the flow sensing component 2 at a predetermined position is not conducted within a predetermined time after the chemical fluid starts to flow.

vi. Although not described in the above detailed embodiments, it should be understood that the three flow sensing components 2 in FIG. 1A can indicate the timing when the chemical fluid enters the flow path R, the timing when the chemical fluid reaches the middle of the flow path R and the timing when the chemical fluid flows out of the flow path R (fully across the entire flow path R), wherein the corresponding information are fed back to the magnetic signal detection chip or detection card, so that the magnetic signal detection chip or detection card can regulate the implementing timing for each processing step.

vii. Although not described in the above detailed embodiments, it should be understood that the detection card may have a part that connects the two second output pads 23 for the same flow sensing component 2 to the electrical circuit, which can be connected to a power source. When the two second output pads 23 of a flow sensing component 2 are electrically conducted by the chemical fluid, the entire electrical circuit is conducted and therefore can output a signal, which can indicate that the chemical fluid flows across the flow sensing component 2.

viii. It should be understood that the number of flow sensing components 2 in a magnetic signal detection chip is not limited to three given in the above embodiment, wherein the number of flow sensing components 2 can be more or less. For example, the number of flow sensing components 2 in a magnetic signal detection chip may be two, and the two flow sensing components 2 may be disposed at both ends of the magnetic signal detection chip in the flow direction F or may be disposed at the downstream end and the central part of the magnetic signal detection chip in the flow direction F. The number of flow sensing components 2 in a magnetic signal detection chip may be one, wherein the one flow sensing component 2 is preferably disposed at the downstream end of the magnetic signal detection chip in the flow direction F.

1. A magnetic signal detection chip, comprising:
a plurality of chemical composition sensing units, which are disposed inside the flow path of the magnetic signal detection chip, through which chemical fluid flows; and
one or more flow sensing component(s), which extend(s) to the outside of the flow path across the flow path, wherein the flow sensing component comprises lead portions and notch portion(s) located between the adjacent lead portions, wherein the notch portion(s) is(are) located in the flow path and at least a part of each lead portion adjacent to the notch portion is exposed to the chemical fluid, so that the flow sensing component(s) is(are) conducted when the chemical fluid completely covers the notch portion(s).

2. The magnetic signal detection chip according to claim 1, wherein the lead portions and the notch portion(s) as a whole extends substantially in an orthogonal direction orthogonal to the flow direction of the chemical fluid.

3. The magnetic signal detection chip according to claim 2, wherein the lead portion comprises interruption/conducting ends at the notch portion, and
in the orthogonal direction, the two outermost interruption/conducting ends of the flow sensing component are located at the boundaries of the flow path or are respectively located at inner side of the corresponding boundary.

4. The magnetic signal detection chip according to claim 1, wherein the flow sensing component comprises only one notch portion, wherein the notch portion is located in the central part in the extending direction of the flow sensing component.

5. The magnetic signal detection chip according to claim 1, wherein the flow sensing component comprises a plurality of notch portions spaced apart from each other, and wherein the plurality of notch portions are substantially uniformly distributed in the flow sensing component in the extending direction of the flow sensing component.

6. The magnetic signal detection chip according to claim 5, wherein the plurality of chemical composition sensing units are arranged in multiple rows, and the arrangement direction of the chemical composition sensing units in each row is along the flow direction of the chemical fluid, and each of the notch portions in the flow sensing component are respectively located in a row in which the chemical composition sensing units are arranged.

7. The magnetic signal detection chip according to claim 1, wherein the flow sensing component further comprises output pads at both ends thereof in an orthogonal direction orthogonal to the flow direction and, wherein the output pads are connected to the lead portions and are disposed outside the flow path.

8. The magnetic signal detection chip according to claim 1, wherein multiple flow detection components are provided, and wherein the multiple flow detection components are arranged in the way of being spaced by the chemical composition sensing units in the flow direction of the chemical fluid, wherein two of the multiple flow detection components are respectively disposed at two ends of the flow path in the flow direction, and/or one of the multiple flow detection components is disposed at the central part of the flow path in the flow direction.

9. The magnetic signal detection chip according to claim 1, wherein one flow detection component is provided, which is disposed at the downstream end of the flow path in the flow direction of the chemical fluid.

10. A detection card, comprising a magnetic signal detection chip comprising:
a plurality of chemical composition sensing units, which are disposed inside the flow path of the magnetic signal detection chip, through which chemical fluid flows; and
one or more flow sensing component(s), which extend(s) to the outside of the flow path across the flow path, wherein the flow sensing component comprises lead portions and notch portion(s) located between the adjacent lead portions, wherein the notch portion(s) is(are) located in the flow path and at least a part of each lead portion adjacent to the notch portion is exposed to the chemical fluid, so that the flow sensing component(s) is(are) conducted when the chemical fluid completely covers the notch portion(s).

11. A nucleic acid detection device, comprising a detection card, which comprising a magnetic signal detection chip comprising:
a plurality of chemical composition sensing units, which are disposed inside the flow path of the magnetic signal detection chip, through which chemical fluid flows; and one or more flow sensing component(s), which extend(s) to the outside of the flow path across the flow path, wherein the flow sensing component comprises lead portions and notch portion(s) located between the adjacent lead portions, wherein the notch portion(s) is(are) located in the flow path and at least a part of each lead portion adjacent to the notch portion is exposed to the chemical fluid, so that the flow sensing component(s) is(are) conducted when the chemical fluid completely covers the notch portion (s).

12. A method of detecting composition containing target DNA fragment in chemical fluid, comprising the following steps:
chemical fluid flowing step, in which during the chemical fluid flowing through a nucleic acid detection device, the chemical fluid flows backwards and forwards in the flow path of a magnetic signal detection chip of the nucleic acid detection device, thereby the chemical fluid completely covers the flow path, so that the composition is fixed to the chemical component sensing unit of the magnetic signal detection chip; and
detecting step, in which the composition containing the target DNA fragment is detected by the magnetic signal detection chip.

13. The method of detecting composition containing target DNA fragment in chemical fluid according to claim 12, wherein the nucleic acid detection device comprises a detection card, which comprising the magnetic signal detection chip comprising:
a plurality of chemical composition sensing units, which are disposed inside the flow path of the magnetic signal detection chip, through which chemical fluid flows; and
one or more flow sensing component(s), which extend(s) to the outside of the flow path across the flow path, wherein the flow sensing component comprises lead portions and notch portion(s) located between the adjacent lead portions, wherein the notch portion(s) is(are) located in the flow path and at least a part of each lead portion adjacent to the notch portion is exposed to the chemical fluid, so that the flow sensing component(s) is(are) conducted when the chemical fluid completely covers the notch portion(s).

14. The method of detecting composition containing target DNA fragment in chemical fluid according to claim 12, wherein the nucleic acid detection device is provided with an inlet portion in communication with the flow path of the magnetic signal detection chip, wherein the chemical fluid flows into the flow path via the inlet portion, and wherein the cross-sectional area of the inlet portion gradually increases towards the flow path.

15. The method of detecting composition containing target DNA fragment in chemical fluid according to claim 14, wherein the width of the part of the inlet portion connected to the flow path is the same as the width of the flow path.

16. The method of detecting composition containing target DNA fragment in chemical fluid according to claim 12, wherein the nucleic acid detection device is provided with an outlet portion in communication with the flow path of the magnetic signal detection chip, wherein the chemical fluid flows away from the flow path via the outlet portion, and wherein the cross-sectional area of the outlet portion gradually decreases from the flow path.

17. The method of detecting composition containing target DNA fragment in chemical fluid according to claim 16, wherein the width of the part of the outlet portion connected to the flow path is the same as the width of the flow path.

18. The method of detecting composition containing target DNA fragment in chemical fluid according to claim 16, wherein the inlet portion and the outlet portion are configured to be symmetrical relative to the centerline of the flow path, wherein the centerline extends along a direction perpendicular to the flow direction of the chemical fluid.
